(19) United States
(12) Patent Application Publication
(10) Pub. No.: US 2022/0020469 A1
(43) Pub. Date: Jan. 20, 2022

(54) SYSTEMS AND METHODS FOR FUNCTIONAL TESTING AND REHABILITATION

(71) Applicant: Children's Hospitals and Clinics of Minnesota, Minneapolis, MN (US)

(72) Inventor: Lynn Renee Tanner, Eagan, MN (US)

(21) Appl. No.: 17/380,559

(22) Filed: Jul. 20, 2021

Related U.S. Application Data
(60) Provisional application No. 63/053,845, filed on Jul. 20, 2020.
(60) Provisional application No. 63/188,675, filed on May 14, 2021.

(51) Int. Cl. G16H 20/30; G16H 10/60; G16H 80/00; A61B 5/11

(52) U.S. Cl. CPC G16H 20/30; G16H 10/60; G16H 80/00; A61B 5/1113; A61B 2562/0219; A61B 2503/06; A61B 2503/04; A61B 2505/09; A61B 2505/07; A61B 5/1128

(57) ABSTRACT

A system and method for providing remote telemedicine, remote rehabilitation or other training is disclosed. Tracking remote physical therapy or training using asynchronous or synchronous feedback between scheduled sessions, where the scheduled sessions can be performed in person or remotely provide for safe and expeditious treatment in situations where in-person sessions cannot be safely conducted and/or when other situations exist making virtual sessions desirable. Tracking subject process between sessions is also accomplished by manual data entry, wearable sensors or video capture by a patient computing device.

### CROSS REFERENCE TO RELATED APPLICATIONS

This application claims the benefit of provisional application Ser. No. 63/053,845 titled “TELEREHABILITATION” filed Jul. 20, 2020, and provisional application Ser. No. 63/188,675 titled “SYSTEMS AND METHODS FOR FUNCTIONAL TESTING AND REHABILITATION” filed May 14, 2021, which applications are incorporated herein by reference in their entirety.

### FIELD OF THE INVENTION

The present invention relates generally to a system and method for providing remote telemedicine, more particularly to providing remote rehabilitation or other training, and more particularly still to a system and method for providing remote physical therapy or training using asynchronous or synchronous feedback between scheduled sessions, where the scheduled sessions can be performed in person or remotely.

### BACKGROUND

Physical therapists (referred to herein as a “PT” or as “PTs” if the context is plural) provide skilled assessments and interventions using hands-on care, prescribed exercises and patient education. Treatments have historically been performed in-person with the PT observing patient progress, modifying the exercises as may be appropriate, and setting goals and treatment plans. Thus, the services are normally accomplished at arranged sessions with a patient in order to optimize the functional impact of treatment and achieve desired outcomes and results. However, due to the limited time periods in which the PT is able to observe the patient, the assessments and exercise recommendations are based on small snapshots of time—e.g., only during the scheduled sessions—and the time periods between sessions are of longer duration.

By observing a patient's functional picture only weekly or monthly, there are significant unobserved periods during which the prescribed exercises should be performed. But oftentimes the patient does not perform the exercises to the prescribed level. In fact, it has been estimated that adherence to these interventions range from 11-80%. If a patient does not complete the exercises, physical therapy services are not as helpful in assisting the patient to achieve an improved physical function.

The rate of progress towards normal developmental motor skills or regaining skills could drastically increase if PTs better understood the adherence to the prescribed exercises, as well as any patient issues or concerns, between therapy visits. Therefore, there is a need in the art for PTs to obtain functional status information between scheduled visits. Having such information would provide the benefit of greater efficiency during in-person or telehealth visits and to maximize a patient's functional status and quality of life.

In a broader context, utilization of telemedicine, particularly for rehabilitation, has historically been very minimal. However, rules and regulations are rapidly changing in response to the COVID-19 pandemic. Medical providers deem rehabilitation to be an essential service, but shortages of personal protective equipment (PPE), scheduling issues, and governmental limits on travel outside the home, among others, demands creative solutions to provide patient care.

Outside of a pandemic, natural disasters and other situations lead to situations in which in-person visits are impossible, inconvenient and/or undesirable. However, use of telerehabilitation in such circumstances may provide greater access to rehabilitation services thereby meeting patient and caregiver needs. Examples of situations in which telemedicine may provide advantages are when multiple patients are cared for in the facility or home, therapists specialized in a type of care are not available in a local community, and/or when patients are unable to travel or are able to interact better in their home environment.

Therefore, there is a need for a system and method to provide information on adherence to exercise, treatment and wellness plans and other events that take place between scheduled visits. Further there is a need in the art for a system and method for rehabilitation using a telemedicine platform.

### SUMMARY

The present invention provides for a system and method for providing enhanced traditional in-person physical therapy treatment (or other training) by providing patient exercise and outcome feedback between sessions. The in-person sessions may also be conducted remotely. Thus, physical therapy, rehabilitation or other training may be improved by utilizing embodiments of a system and method for delivering remote physical therapy or training services. A further advantage of such a system is providing asynchronous or synchronous feedback between the scheduled sessions. By providing feedback on the number, time and rate of exercises and events between scheduled sessions, the physical therapy and training may be modified to improve results and modify patient outcomes and plans. In the event that remote sessions are required or preferred, existing online and internet enabled video session technology may be used. For example, products and apps such as ZOOM®, WEBEX®, Facetime®, and others are commercially available.

A level of data concerning the patient's physical function, exercise adherence, and the functional variability experienced between physical therapy visits is beneficial. More specifically, the information allows the therapist to provide accurate and efficient recommendations for the individual patient thereby leading to better outcomes.

In the case of a younger patient, both the patient and family can be empowered to engage in the physical therapy recommendations—thereby progressing at a faster rate. The family also benefit from this day to day objective information to see progress—or regression where they may need a PT's assistance. The ability to connect exercise adherence to functional progress helps create accountability within the child, caregiver, and therapist relationship. Both the therapist and family benefit with this knowledge to optimize physical function throughout the treatment.

Therefore, according to one aspect of the invention, there is provided a system for providing functional testing and training of a patient, the system comprising: at least one patient computing device comprising a processor and a memory comprising instructions that, when executed, operate a functional testing application comprising: a functional testing service configured to manage a telerehabilitation functional testing session with the patient; a goal setting service configured to create and monitor functional fitness goals for the patient; a home exercise service configured to guide the patient through exercises selected for the patient; a progress reporting service configured to track data collected by the functional testing service and the home exercise service to track rehabilitation progress of the patient; and a provider communication service configured to facilitate communication between the patient and a healthcare provider.

In accordance with the previous paragraph, the system may further include one or more sensors configured to be worn by the patient, the one or more sensors operating to record movements of the patient and transmit data regarding the movements to the at least one patient computing device and/or wherein the provider communication service is further configured to facilitate communication with a proxy of the patient.

In accordance with another aspect of the invention, there is provided a non-transitory computer-readable storage medium comprising computer-executable instructions which, when executed by a computing system, cause the computing system to perform a method of functional testing of a subject, the method comprising: receiving login information from the subject at a mobile computing device; presenting prompts on the mobile computing device to have the subject perform one or more actions; record subject movements with the mobile computing device; analyze movements to provide functional data regarding the subject's physical function capacity; and communicate the functional data to a computing device operated by an observer.

In accordance with the previous paragraph, the subject may be a pediatric patient and the observer may be a medical professional.

In accordance with the previous paragraph, the method may include, wherein the patient movements are recorded using an accelerometer; wherein the movements are analyzed by comparing the patient movements to standard movements corresponding to the prompts; and/or wherein the movements are analyzed in real time as the patient performs the movements.

In accordance to yet another aspect of the invention, there is provided a telerehabilitation system comprising: at least one patient computing device comprising a video conferencing application configured to receive telerehabilitation services; at least one telemedicine provider computing device configured to operate a video conferencing application to communicate with the at least one patient computing device to provide telerehabilitation services; a telemedicine server comprising: a graphical user interface operable to present a visual display on the at least one patient computing device and at least one telemedicine provider computing device for facilitating telerehabilitation sessions; a communication module operable to manage communication between the at least one patient computing device, the at least one telemedicine provider computing device, and the telemedicine server; a telerehabilitation services manager operable to coordinate telerehabilitation services; and a data store comprising telerehabilitation resources accessible by the at least one patient computing device and the at least one telemedicine provider computing device.

According to still another aspect of the invention, there is provided a system for providing remote rehabilitation therapy, comprising: one or more sensors configured to be worn by a patient, the sensors comprising: movement sensors configured to record motion data from inertial and/or mechanomyographic movements of a patient during an activity; and a wireless communication device; a computing device comprising: a processing device; and a memory device comprising instructions which, when executed by a computing device, cause the computing system to: receive, via wireless communication, the motion data recorded by the sensors; communicate the motion data to a computing device operated by a medical professional.

These and other advantages and features which characterize the present invention are pointed out with particularity in the claims annexed hereto and forming a further part hereof. However, for a better understanding of the invention, the advantages and objects obtained by its use, reference should be made to the drawings which form a further part hereof, and to the accompanying descriptive matter in which there is illustrated and described a preferred embodiment of the present invention.

### DESCRIPTION OF THE DRAWINGS

The following drawing figures, which form a part of this application, are illustrative of described technology and are not meant to limit the scope of the disclosure in any manner.

FIG. 1 shows a schematic diagram of an example telerehabilitation system in which the principles of the present invention may be employed.

FIG. 2 shows a schematic diagram of the functional testing application of FIG. 1.

FIG. 3 shows a schematic diagram of the telemedicine server of FIG. 1.

FIG. 4 is a schematic diagram of the physical components of an example computing device usable in the healthcare environment of FIG. 1.

FIG. 5 illustrates a decision tree for determining whether to provide telerehabilitation to an outpatient and determine a plan of treatment.

FIG. 6 illustrates an example decision tree for determining how to handle telemedicine visits for patients requiring inpatient physical therapy and/or occupational therapy during a pandemic.

FIG. 7 illustrates an example decision tree for determining how to handle telemedicine visits for patients requiring physical therapy, speech-language pathology, and/or occupational therapy during other times.

FIG. 8 illustrates an example decision tree for determining how to handle telemedicine visits for patients requiring inpatient speech therapy.

FIG. 9 is a logical flow diagram illustrating method steps which may be used in connection with the present invention.

### DETAILED DESCRIPTION

Various embodiments of the present disclosure will be described in detail with reference to the drawings, wherein like reference numerals represent like parts and assemblies throughout the several views. Additionally, any examples set forth in this specification are not intended to be limiting and merely set forth some of many possible embodiments.

Broadly, the present disclosure is directed to systems and methods for managing patient rehabilitation with at-home physical therapy and functional testing performed with the aid of a mobile device or other computing device. A computing application operable on a patient computing device such as a smartphone or tablet is configured to test physical function, monitor progress, and communicate this with the medical team when appropriate. A pediatric patient can hold a mobile computing device (e.g., an iPhone®, Android® operating system phone, tablet or other similar smart devices) and complete functional tests in their own home to provide functional data to demonstrate physical function capacity.

This application could be used to track improvements related to rehabilitation services in order to match or exceed the functional outcomes of in-person rehabilitation. This application could be used in well-child checks to indicate overall cardiovascular/developmental health. The app could include 3-7-minute workouts that are pediatric-based and track changes with tests. The application could be used to motivate child as the app would show progress over time. Gamification can provide further motivation to utilize the application. Possible users for such an application include: pediatrics clinics, pediatric hospitals, rehabilitation clinics, families, sporting coaches/clubs, fitness clubs, personal trainers, and adults using rehabilitation.

Various embodiments described in the present disclosure use the example of physical therapy services. However, other rehabilitation services can use the same or similar methods/system and are also within the scope of this disclosure. For example, occupational therapy or speech therapy could be performed with a similar application operating on a computing device. Similarly, a personal trainer may track the progress of a an individual with whom they are working.

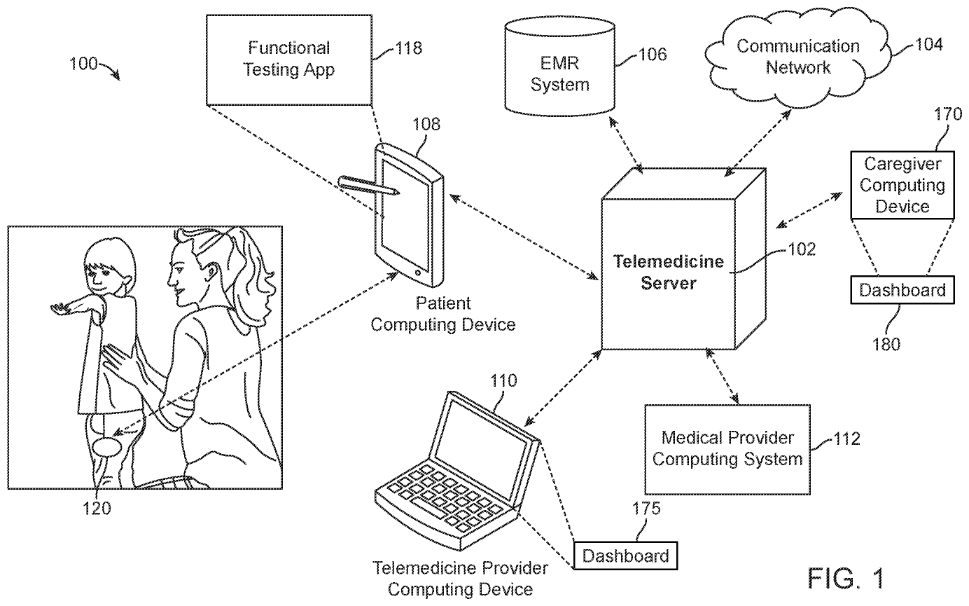

FIG. 1 is a schematic diagram of an example system 100 for providing functional testing and training for patients. The system 100 facilitates communications between patients and healthcare practitioners, manages healthcare data relating to functional testing, and provides electronic resources to healthcare practitioners. The system 100 includes a server 102, a communication network 104, an electronic medical record system 106, a patient computing device 108, a physical therapist provider computing device 110, and a medical provider computing system 112. Each component of this system 100 communicates with each other via wired or wireless connections. In some embodiments, additional components can communicate with the system 100 through the communication network 104. In some embodiments, some of the components shown in FIG. 1 are optional.

The server 102 operates to manage communications and data relating to operation of the functional testing application 118. In some embodiments, various types of data can be stored at the server 102. In some embodiments, the functional testing application 118 is hosted on the server 102 and is accessed by the patient computing device 108.

One or more components of the system 100 are in communication with each other via a communication network 104. The communication network 104 may include any type of wireless network, a wired network, or any communication network known in the art. For example, wireless connections can include cellular network connections and connections made using protocols such as 802.11a, 802.11g, 802.11n, and/or 802.11ac.

The electronic medical record (EMR) system 106 operates to record and manage electronic medical records for patients of one or more medical facilities. In some embodiments, the EMR system 106 includes files relating to rehabilitation of patients.

The patient computing device 108 and physical therapist computing device 110 operate to communicate with one another through the communication network 104. Video communication software can operate on the computing devices to allow a healthcare practitioner to demonstrate rehabilitation therapies to a patient and to observe a patient's movements to complete rehabilitation therapies. Additionally, text or voice communications on a synchronous or asynchronous basis can be managed through the communication network 104.

The functional testing application 118 is configured to provide tests that can be performed by a pediatric patient with a portable computing device to assess various physical abilities of the patient. The functional testing application 118 can be accessed by the patient computing device 108 to access workouts and submit results of testing. The functional testing application 118 can also be accessed by the PT computing device 110 to manage patient care. Dashboard 175 operating on the provider computing device 110 (or web based) may provide updates and parameters on the patient's adherence to a treatment plan. Such information may include information on the time, date, length, duration, number of reps, and other parameters of a rehabilitation plan. In addition, the dashboard 175 may include a listing of a number of patients of the PT, wherein selection of one of the elements of the list provides the detailed report for the individual patient. In some embodiments, the functional testing application 118 is hosted on the server 102 for access by multiple computing devices. Additional details regarding the functional testing application 118 are provided later in this document.

In some embodiments, the patient computing device 108 communicates with one or more sensors 120 worn by a patient during a training or testing session. In some embodiments, the sensors are usable to record movements of a patient. The movements can be used in conjunction with the functional testing application 118 or during telerehabilitation sessions.

Also shown in FIG. 1 is caregiver computing device 170 which also communicates with the telemedicine servicer 102. Caregiver computing device 170 may be used by families and other caregivers to monitor the progress of the patient and may include a dashboard 180 which may provide information on the time, date, length, duration, number of reps, and other parameters of a rehabilitation plan.

The medical provider computing system 112 operates to communicate with the server 102 to access EMRs, access resources for telemedicine, and schedule telerehabilitation sessions for patients and HCPs.

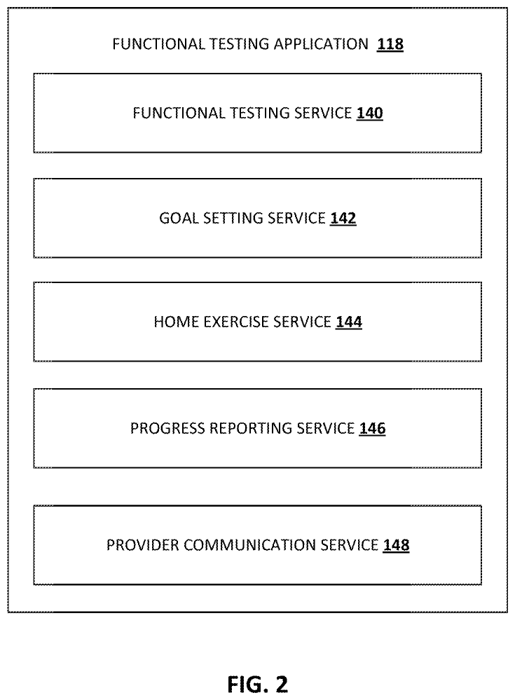

FIG. 2 shows an example functional testing application 118. In some examples the functional testing application 118 includes a functional testing service 140, goal setting service 142, home exercise service 144, progress reporting service 146, and provider communication service. In some examples, the functional testing application 118 is used to supplement rehabilitation care as well as provide consumers and HCP objective physical performance data captured by a mobile device, such as patient computing device 108 illustrated in FIG. 1.

The functional testing application 118 can be used in a variety of health care settings ranging from pediatric to adult care. It can be used in conjunction to rehabilitation care or could be used in the preventative care setting to provide objective information. It could also be used to test athletic performance with general home exercise programs for the general consumer. In some examples, gamification is used to enhance use and adherence to a rehabilitation plan. Additionally, the functional testing application 118 connects exercise adherence to functional progress. This feature allows the application to create consistency with a patient's treatment between a child, caregiver, and therapist. In some examples the AI is trained, in full or part, with data from clinical trials related to the rehabilitation treatment.

The functional testing application 118 includes a functional testing service 140. The functional testing service 140 manages a functional testing session with a patient. Examples of tests in a functional testing session include tests for determining walking capacity, safe home mobility, running and agility, balance, strength, and flexibility. The functional testing application 118 provides instructions to complete functional testing test, monitors the functional tests, and records data from the test. In some examples, the functional tests are monitored by a provider using the provider communication service 148. In some examples, videos are provided to a patient, or a caregiver, to instruct the patient on exercises needed to complete the functional testing. In some examples the functional testing service 140 collects objective numerical data and video. In some examples, this service is managed by artificial intelligence (AI), which automatically selects, monitors, and/or manages a functional testing session. In some examples the AI is trained, in full or part, with data from clinical trials related to the rehabilitation treatment.

In some embodiments, the functional testing application 118 includes a goal setting service 142. The goal setting service 142 is used by rehabilitation patients and providers to create progress goals for a patient. In some examples, these goals are based on the results of a functional test. The goal setting service 142 can link the functional testing service 140 with the home exercise service 144. For example, a patient may have weekly functional testing sessions and based on the results of these sessions, the goals set for the patient, and the progress reporting service 146 a home exercise plan may be created by the home exercise service 144. In some examples, this service is managed by artificial intelligence, which automatically recommends and selects goals based on a child's progress. In some examples the AI is trained, in full or part, with data from clinical trials related to the rehabilitation treatment.

In some embodiments, the functional testing application 118 includes a home exercise service 144. The home exercise service 144 provides exercises to a patient. In some examples the exercises are automatically selected based on the patients functional testing results, goals, and progress reports. In other examples, a rehabilitation expert selects the home exercises. In some examples, the exercises are monitored either by data collected on the mobile device, by a provider using the provider communication service 148, or both. In some examples, videos giving instructions on the home exercises are provided to the patient or caregiver. In some examples the home exercise service 144 collects objective numerical data and video. In some examples, this service is managed by artificial intelligence, to provide home exercises which allows a patient to meet their goals. In some examples, the application may include comparisons to other children, or ways to communicate with other patients in a virtual social setting, with proper permissions required.

Generally, the exercise program can be selected by a physical therapist for a patient. In some embodiments, videos illustrating exercises can be selected using the PT computing device 110. In some embodiments, an existing exercise routine can be selected and modified by the PT. As the patient performs the exercises, the application operating on the patient computing device 108 records the patient's progress and completion of the program. In some embodiments, the functional testing application 118 can provide reminders to the patient on the patient computing device 108 to perform the exercise routine selected by the PT.

The application can include a series of 3-7 min workouts to target the testing areas if a patient wants to make improvements. The workouts are age appropriate (5-7, 8-10, 11-18) and are linked to the test result. For example, if the patient is weak on the 30 second sit to stand exercise, the application would link to a workout that is relatively easy to improve that testing area. If the patient performs fair on the sit to stand test, the application links to a harder workout.

The following are some examples of tests that may be performed using the application:
30 sec sit to stand (chair stand test)
Timed up and down stairs test (TUDS)
Timed up and go test (TUG)
6-minute walk test
Broad jump distance
Single leg stance eyes open/closed
Tandem eyes open/closed
Running speed—20 meter shuttle run test
Hamstring, ankle, shoulder flexibility

In some embodiments, the application can provide athlete related tests such as the triple hop and PACER. In some embodiments, these tests require a connection to a Bluetooth device worn by the patient.

In some embodiments, the application can share results of the functional testing with a patient's EMR. In some embodiments, the results are shared with a caregiver or parent user of the application to review the results (e.g., on caregiver computing device 170). In some embodiments, the results are displayed compared to childhood norms for the patient's age.

In some embodiments, the functional testing application 118 includes a progress reporting service 146. The progress reporting service 146 tracks the data collected by the functional testing service and home exercise service to track the rehabilitation progress for a patient. In some examples, the progress reporting service 146 will also receive inputs from a rehabilitation expert. Additionally, the progress reporting service 146 may sync with electronic medical records (EMR) to provide up-to-date status for a patient. Sharing data with an EMR service requires the application to receive permission from the patient and/or caregiver.

In some embodiments, the functional testing application 118 includes a provider communication service 148. The provider communication service 148 can include synchronous or asynchronous communication. For example, the provider communication service 148 can provide live video or audio conference calling. In other examples, the provider communication may be recorded video, or text. In some examples, a patient or care provider selects a communication preference. The provider communication service 148 allows a healthcare provider to give feedback, instructions, suggestions, and rehabilitation expertise to a patient or caregiver.

The graphical user interface (GUI) 150 operates to present a visual display for computing device users to access the functional testing application 118. In some embodiments, the GUI 150 presents an interface to a patient on the patient computing device 108 for accessing recommended exercises, performing functional testing, and communicating with a healthcare provider (HCP). The GUI 150 presented to the HCP could display different options than the GUI 150 presented to the patient. For example, on the physical therapist computing device 110, the GUI could include the dashboard 175 as well as showing options for creating workouts, viewing the results of a patient's functional testing, and communicating with patients.

In some examples, the functional testing application 118 includes a GUI designed for a rehabilitation specialist perspective. From this perspective, the specialist is able to modify the exercise recommendations throughout a variable patient status. In this manner the specialist is able to rely on objective data, live video, video recordings or other methods to evaluate a patient. The specialist is also able to make more frequent updates to a rehabilitation program, which can allow the patient to develop faster. The application also allows a specialist to instruct a caregiver or patient on what to do next to continue making progress. Additionally, the specialist can use the functional testing application 118 to supplement synchronous rehabilitation visits.

For example, the functional testing application 118 allows a physical therapist (PT) to provide exercise recommendations with greater precision. This precision will create greater trust in the rehabilitation process creating greater home exercise adherence. The functional variety of data from this mobile application will increase PT visit efficiency with the data identifying the areas of needed reassessment allowing more time for individual tailoring of a visit that requires the skill of a trained PT.

In some examples, the various services may include additional features or user interfaces based on characteristics of the patient. For example, features may include gamification for children. The gamification may be optimized for a child or a certain age and require supervision for some social aspects. In contrast, an adult version can include broader permissions and features optimized for adults.

Different patient interfaces can be presented to different audiences. For example, a child's patient interface could be different from a teen's patient interface. A parent or caregiver interface would be different from a patient interface. The child interface would be gamified to be fun and whimsical with a challenge and “badges” when change is made, the teen interface could be gamified at a different level showing a graph of improvement or something, and the parent interface could show how the results compare to the norms for the child's age group and the progress made. In some embodiments, a “travel across the world” game includes a map and info about what kids do in a different country when they arrive at the milestone. The patient can then track their progress across the world. For teen users, their steps (if using their phone) can be used to virtually travel across the world in some way with ability to “jump a country” if they improve their function test scores.

The functional testing application 118 provides many benefits to patients and care providers. For example, families and patients often struggle providing beneficial exercises outside of visits to rehabilitation specialists. The functional testing application 118 can also provide better understanding of a child's home physical function, exercise adherence, and the functional variability the patient experiences between visits, to a specialist. This information allows the therapist to provide accurate and efficient recommendations for the individual family in order to empower them to engage in the PT recommendations to progress at a faster rate. The family would also benefit from this day-to-day objective information to see progress or see regression where they may need a PT's assistance. The functional testing application 118 is caregiver-friendly and mobile. The functional testing application 118 further allows remote monitoring/communication of functional status.

In some embodiments, one or more features of the application can be included in a free version of the application and other features are only accessible for users that pay for a subscription or a paid version of the application.

In some embodiments, the application requires the user to sign a waiver before use. In some embodiments, the waiver can be signed electronically.

In some embodiments, the testing and training described above can be performed using wearable sensors such as the wearable sensors 120 of FIG. 1. These sensors will allow greater objectivity in the virtual setting to maximize efficacy of physical therapy and functional testing compared to in-person visits in order to provide value-based care. The sensors can be embedded in clothing that the child wears during therapy or during testing. In some embodiments, the sensors are integrated into shirts and/or pants. In some embodiments, the sensors clip onto clothing of the patient. In some embodiments, other wearable sensors are possible. In some embodiments, the sensors communicate with a patient computing device using Bluetooth or other wireless communication technology. The sensors are usable to capture inertial and/or mechanomyogram signals (among others) indicative of the physical movements of the patient.

In some embodiments, the sensors can be used with a home exercise program that integrates video with wearable sensors. A program including daily physical activity classes for different ages and levels can be provided. In some embodiments, there is social interaction and gamification. In some embodiments, the patient's level for the classes is determined by a physical therapist.

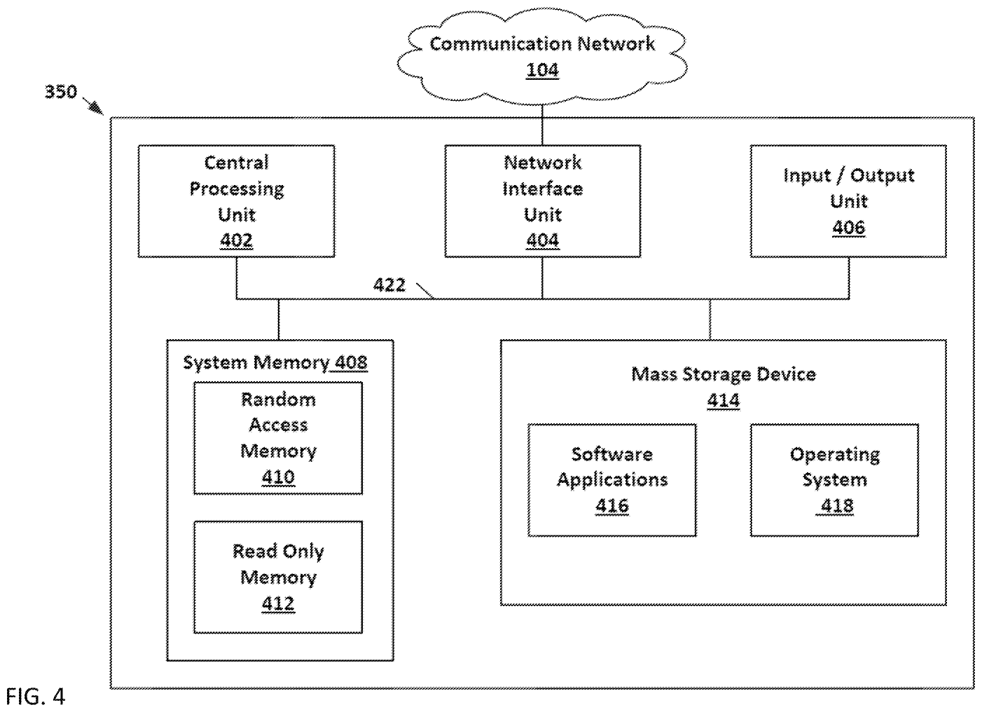

In the example shown in FIG. 4, the computing device 350 includes at least one central processing unit (“CPU”) 402, a system memory 408, and a system bus 422 that couples the system memory 408 to the CPU 402. The system memory 408 includes a random access memory (“RAM”) 410 and a read-only memory (“ROM”) 412. A basic input/output system that contains the basic routines that help to transfer information between elements within the computing device 350, such as during startup, is stored in the ROM 412. The computing device 350 further includes a mass storage device 414. The mass storage device 414 is able to store software instructions and data.

The mass storage device 414 is connected to the CPU 402 through a mass storage controller (not shown) connected to the system bus 422. The mass storage device 414 and its associated computer-readable storage media provide non-volatile, non-transitory data storage for the computing device 350. Although the description of computer-readable storage media contained herein refers to a mass storage device, such as a hard disk or solid state disk, it should be appreciated by those skilled in the art that computer-readable data storage media can include any available tangible, physical device or article of manufacture from which the CPU 402 can read data and/or instructions. In certain embodiments, the computer-readable storage media comprises entirely non-transitory media.

Computer-readable storage media includes volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the computing device 350.

According to various embodiments, the computing device 350 can operate in a networked environment using logical connections to remote network devices through a communication network 104, such as a wireless network, the Internet, or another type of network. The computing device 350 may connect to the communication network 104 through a network interface unit 404 connected to the system bus 422. It should be appreciated that the network interface unit 404 may also be utilized to connect to other types of networks and remote computing systems. The computing device 350 also includes an input/output controller 406 for receiving and processing input from a number of other devices, including a touch user interface display screen, or another type of input device. Similarly, the input/output controller 406 may provide output to a touch user interface display screen or other type of output device.

As mentioned briefly above, the mass storage device 414 and the RAM 410 of the computing device 350 can store software instructions and data. The software instructions include an operating system 418 suitable for controlling the operation of the computing device 350. The mass storage device 414 and/or the RAM 410 also store software instructions, that when executed by the CPU 402, cause the computing device 350 to provide the functionality discussed in this document.

### ALTERNATIVE EMBODIMENT

The present disclosure is further directed to systems and methods for managing various forms of patient care, including rehabilitation, performed through telemedicine. In some embodiments, inpatient rehabilitation is performed with the patient in a medical facility, but in a different room from the healthcare practitioner. In some embodiments, outpatient rehabilitation is performed with the patient at home and the healthcare practitioner at a medical facility (or location remote from the patient). In all instances, a video communication system is utilized to facilitate interactions between the patient and the healthcare practitioner. Such systems may lead to a decrease in the costs associated with medical visits to patients (travel, missing work) and increased patient convenience. Additionally, such a system may help decrease facility costs to the hospitals. Still further, it would allow specialized rehabilitation providers to reach children out of their region and provide rehabilitation with greater accuracy using wearable technology.

While FIG. 1 is discussed above, a brief discussion in the broader context of telerehabilitation and is next provided. FIG. 1 provides a schematic diagram of an example telerehabilitation system 100. “Telerehabilitation” refers to the process of a healthcare practitioner providing rehabilitation services to a patient using a telecommunication platform. The telerehabilitation system 100 facilitates communications between patients and healthcare practitioners, manages healthcare data relating to telerehabilitation services, and provides electronic resources to healthcare practitioners. As discussed above, the telerehabilitation system 100 includes a telemedicine server 102, a communication network 104, an electronic medical record system 106, a patient computing device 108, a telemedicine provider computing device 110, and a medical provider computing system 112. Each component of this healthcare environment 100 communicates with each other via wired or wireless connections. In some embodiments, additional components can communicate with the healthcare environment 100 through the communication network 104. In some embodiments, some of the components shown in FIG. 1 are optional.

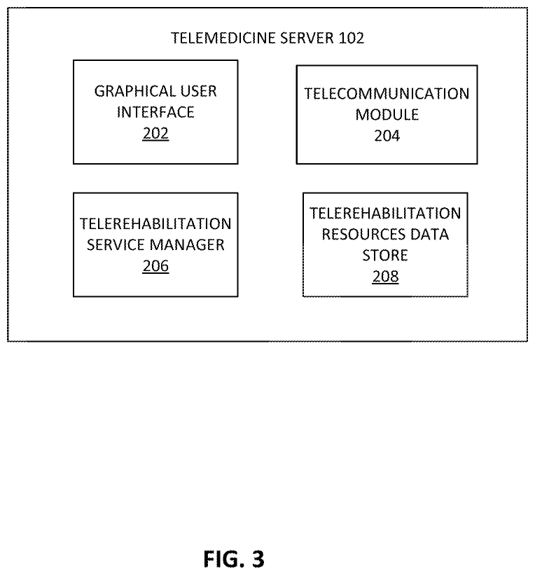

The telemedicine server 102 operates to manage communications and data relating to telerehabilitation services. The telemedicine server 102 is described with greater detail in FIG. 3.

One or more components of environment 100 are in communication with each other via a communication network 104. The communication network 104 may include any type of wireless network, a wired network, or any communication network known in the art. For example, wireless connections can include cellular network connections and connections made using protocols such as 802.11a, 802.11g, 802.11n, and/or 802.11ac.

The electronic medical record (EMR) system 106 operates to record and manage electronic medical records for patients of one or more medical facilities. In some embodiments, the EMR system 106 includes files relating to rehabilitation of patients.

The patient computing device 108 and telemedicine provider computing device 110 operate to communicate with one another through the communication network 104. Video communication software operates on the computing devices to allow a healthcare practitioner to demonstrate rehabilitation therapies to a patient and to observe a patient's movements to complete rehabilitation therapies.

In some embodiments, the patient computing device 108 also operates a functional testing application 118. The functional testing application 118 is configured to provide tests that can be performed by a pediatric patient with a portable computing device to assess various physical abilities of the patient. Additional details regarding the functional testing application 118 are provided later in this document.

In some embodiments, the patient computing device 108 communicates with one or more sensors 120 worn by a patient during a telerehabilitation session. In some embodiments, the sensors are usable to record movements of a patient. The movements can be used in conjunction with the functional testing application 118 or during telerehabilitation sessions. Further details regarding the use of sensors are provided later in this document.

The medical provider computing system 112 operates to communicate with the telemedicine server 102 to access EMRs, access resources for telemedicine, and schedule telerehabilitation sessions for patients and HCPs.

FIG. 3 is a more detailed schematic diagram of the telemedicine server 102 of FIG. 1. The telemedicine server 102 includes a graphical user interface (GUI) 202, a telecommunication module 204, a telerehabilitation services manager 206, and a telerehabilitation resources data store 208.

The graphical user interface (GUI) 202 operates to present a visual display for computing device users to access a telecommunication system for receiving or providing telerehabilitation services. In some embodiments, the GUI 202 presents an interface to a patient to log into a software service to communicate with an HCP. The GUI 202 presents various options relating to communication with a HCP, scheduling appointments, providing patient data to a healthcare facility, and receiving instructions for completing rehabilitation at a location remote from a HCP. The GUI 202 presented to the HCP could display different options than the GUI 202 presented to the patient.

The communication module 204 operates to manage communication between patient computing devices 108, telemedicine provider computing devices 110, and the telemedicine server 102. In some embodiments, the communication module 204 further facilitates communication with an EMR system 106 and a medical provider computing system 112.

The telerehabilitation services manager 206 operates to coordinate various aspects of providing telerehabilitation services to patients. For example, the telerehabilitation services manager 206 can provide electronic scheduling of patients with particular HCPs for video conference therapy sessions. In some embodiments, the telerehabilitation services manager 206 provides telecommunications information for a session along with EMR information for a patient to a HCP before an upcoming scheduled rehabilitation session. In some embodiments, the telerehabilitation services manager 206 provides reports regarding rehabilitation sessions to an EMR system 106, as well as other computing systems such as an insurance payor insurance computing system.

The telerehabilitation resources data store 208 operates to store information regarding informational resources usable by an HCP or a patient. In some embodiments, the telerehabilitation resources data store 208 includes instructions for best practices in providing telerehabilitation, decision trees, troubleshooting guides, and recommendations for providing treatment to particular types of patients. In some embodiments, the telerehabilitation resources data store 208 also provides resources to patients for completing rehabilitation such as guides for completing rehabilitation at home without guidance of a HCP, instructions for utilizing telecommunication systems, and instructions for completing rehabilitation exercises with or without an HCP.

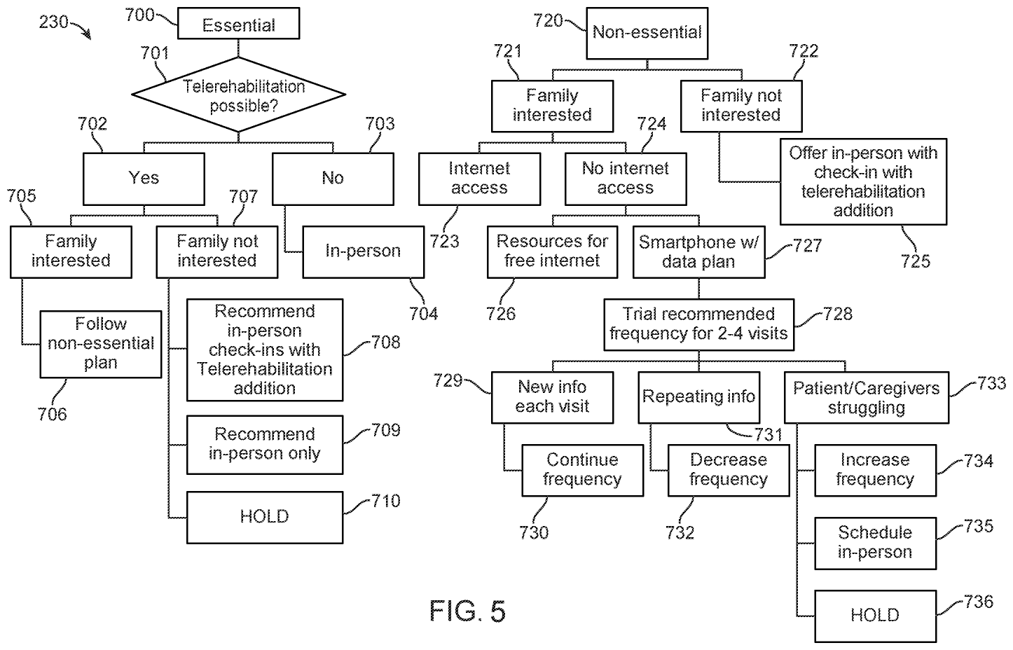

Turning now to FIG. 5 a logical flow is illustrated generally at 230 for determining whether to provide telerehabilitation to a patient and determine a plan of treatment. The process starts at block 700 where it is determined whether the rehabilitation is essential or nonessential. If essential, the process moves to block 701 where it is determined whether tele-rehabilitation is possible. If yes, the process continues to block 702 and then moves to a discussion with family (or other caregiver; referred to collectively as “family” herein unless the context makes it clear that it excludes other caregivers) to determine if there is interest (blocks 705 and 707). If the family is interested process moves to block 706. At that time, the “non-essential” telerehabilitation process is then reviewed at block 720. If the family is not interested, then the process proceeds from block 707 to blocks 708, 709, and 710. At block 708 recommending in-person check-ins with telerehabilitation additions is considered; at block 709 recommending only in-person visits are considered; and at block 709 no immediate decision is reached.

Next at block 720 “non-essential” rehabilitation services are considered with the process moving to block 721 to determine whether the family is interested or to block 722 if the family is not interested. If interested, the process determines whether Internet access is available at blocks 723 and 724. If there is internet access, then telerehabilitation services can begin at block 724. If there is no internet access, then the process moves from block 724 to determine if there are resources for free internet access at block 726 or if a smartphone with a data plan is available at block 727. In the event that only a smart phone is available, at block 728 a trial is recommended for two to four visits (i.e., to gauge the effectiveness of the service through use of the device). At block 729, it is determined whether new info should be provided for each visit and the visits continue with the same frequency at block 730. Alternatively, at block 731, it is determined whether repeating info is needed and the frequency should be decreased at block 732. Finally, at block 733 it is determined if the patient and/or family are struggling. In that case, then the process moves to determine if increased frequency is needed at block 734; scheduling in-person visits are needed at block 735; and/or no immediate decision is made at block 736.

Table 1 below provides an example of the elements to be considered and reviewed as part of evaluating a telerehabilitation evaluation session.

TABLE 1
Telerehabilitation Evaluation Session Overview
1. Client history
EMR review
Parent interview
Medical history
Developmental history
Reason for referral/Caregiver
concerns and goals
2. Assure set-up for session
Review pre-evaluation standardized
questionnaire if applicable
Equipment
Space
Camera position
3. Objective assessment
Clinical observation
Standardized assessment
Non-standardized assessments
4. Assessment/plan
Share assessment results with caregiver
Determine need for future services
Educate family on treatment diagnosis
and future plan of care
Educate and demonstrate initial
home program
5. Connect family with scheduling
team for future visits
6. Brief joint planning for next session
Activities
Space
Equipment materials

Table 2 below provides an example of the elements to be considered and reviewed as part of evaluating a telerehabilitation treatment session.

TABLE 2
Telerehabilitation Treatment Session Overview
1. Status review
Review symptoms
Review progress towards goals
Questions from last session
Home programming update
Concerns for day's session
2. Assure set-up for session
Activities
Equipment
Space
Camera position
3. Therapeutic activities/Therapeutic
Goal assessment
Demonstrate/explain first especially
if new activity
Physical demonstration
Doll
Video/image
Practice together
Provide verbal cues and instruct
caregiver on techniques
Facilitation
Inhibition
Adaptation
Compensation
Assess activity performance
Reflect with caregiver
Use parent coaching or motivational
interviewing techniques
Therapist give feedback
Assessment/plan
Correct compensations
Give strategies to elicit improved performance
Progress or modify/adapt activity to
fit day's performance
4. Brief joint planning for next session
Activities
Space
Equipment materials
Date/time

Various barriers may arise, making it difficult to provide telerehabilitation services. Table 3 below provides guidance for HCPs on how to address those difficulties.

TABLE 3

| Barriers | Possible Solutions |
|---|---|
| Autism Spectrum | Child-directed model |
| Disorders | Observation in natural environment |
| | Focus on parent coaching |
| Behavior/short | Shorter, more frequent sessions |
| attention span | Movement breaks, during that time |
| | focus on caregiver education |
| | Use of videos and music to engage child |
| | Involve siblings, when appropriate |
| | Ensure that patient has tangible motivators |
| | available (e.g. stickers, snacks) |
| Skilled handling | Creative functional treatment activities/goals |
| required | Step-by-step parent coaching over sessions |
| | In-person may be required |
| Light/screen | Dimmed screen |
| sensitivity | Have patient turn away from |
| | screen as needed |
| | In-person may be required |
| Vital Stim | In-person sessions |
| therapy, concern | |
| for aspiration | |
| CI patients, | Trial Telerehabilitation, with use of |
| aided/unaided | proper audio equipment |
| hearing loss | In-person visits may be required |
| Necessity for vitals | Fitbit or home tech available |
| | RPE scale |
| | Teens taking own HR |
| Tuning orthotics | Mail tuning objects to them |
| Lack of | Use of household objects for toys |
| toys/supplies/ | Using music/singing |
| resources | Apps for visual motor skills |
| | Sidewalk chalk outside |
| | Use of nature |
| Balance/safety | Minimize obstacles |
| | Strategic placement of furniture/support |
| | Use of corners for support |
| | Caregiver presence |
| Manual Therapy | Teach self-palpation strategies |
| | Teach caregiver |
| | In-person may be required |
| Complex gait | Sharing videos to allow analysis |
| | Recording virtual session with |
| | patient permission (using e.g. |
| | Amwell telehealth application) |

Table 4 below provides parameters and issues that may be considered on whether to provide inpatient telerehabilitation services.

TABLE 4
Inpatient Telerehabilitation
Patient Selection
During a pandemic, there may be a need to
reduce contact with caregivers in order to
reduce the need for personal protective
equipment (PPE) and reduce transmission
of disease
Telerehabilitation should be used when one
or more of the following apply:
Strong caregiver presence
Older child
Stable course or safe plan
Difference between some rehab or none at all
Instead of telerehabilitation, reduced in-person
frequency should be used when one or more
of the following apply:
Limited caregiver presence
Caregiver barriers
Behavioral challenges
Unable to manage lines
Nursing unavailable
Change in status expected

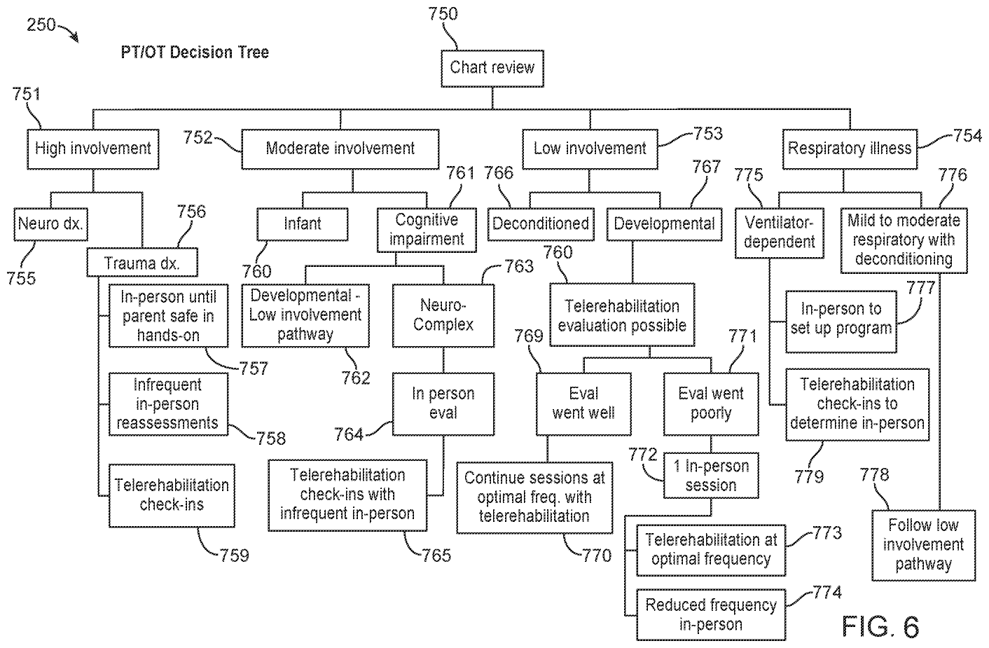

FIG. 6 illustrates an example decision tree 250 for determining how to handle telemedicine visits for patients requiring physical therapy (PT) and/or occupational therapy (OT) in the inpatient setting during a pandemic or health crisis.

Now referring to FIG. 6, a representative physical therapy and occupational therapy logic flow for the provision of telerehabilitation services is provided. The logical flow is shown generally at 250 and begins at block 750 with a review of a patient's chart. The chart is reviewed to determine the level of involvement with high involvement at block 751; moderate involvement at block 752; low involvement at block 753, or respiratory illness at block 754. In the event that high involvement is determined, the diagnosis is then reviewed to determine whether there is a neurological diagnosis at block 755 or a trauma diagnosis at block 756. In the case of a neurological diagnosis, other processes to move forward are utilized. However, if a trauma diagnosis is determined, then the process moves at block 757 to provide in-person sessions until the parents are comfortable with hands-on sessions; infrequent in-person reassessments are provided at block 758; and tele-rehabilitation check-ins take place at block 759.

Still referring to FIG. 6, if moderate involvement is determined at block 752, then the process moves to block 760 to determine whether it is an infant. If so, other necessary processes are then required. If there is cognitive impairment determined at block 761, then it is decided whether there is a developmental low involvement pathway at block 762 or neural complex at block 763. In the event of the neural complex, the process moves to block 764 for an in-person evaluation and followed, at block 765, with telerehabilitation check-ins with infrequent in-person sessions.

If low involvement is determined at block 753, the next step is to determine whether it is deconditioned at block 766 or a developmental issue at block 767. At block 766 other processes forward are taken. If it is a developmental issue at block 767 then a telerehabilitation evaluation is possible. If the evaluation goes well at block 769, then sessions are continued at block 776 on a rehabilitation basis at an optimal frequency. However, if the evaluation went poorly at block 771, then one in-person session is scheduled at block 772 to determine whether telerehabilitation at optimal frequencies should be scheduled (block 773) and/or whether reduced frequency in-person visits should be scheduled at block 774.

At block 754 if respiratory illness is determined, then the process moves to block 775 to determine whether the patient is ventilator dependent. If the answer is yes, then at block 777 an in-person visit is scheduled to set up the program and telerehabilitation check-ins are scheduled to determine whether and when to schedule in-person visits at block 779. If the respiratory illness is not ventilator dependent at 775 the process instead moves to block 776 where mild to moderate respiratory with deconditioning is reviewed. The process then moves to a low involvement pathway at block 778.

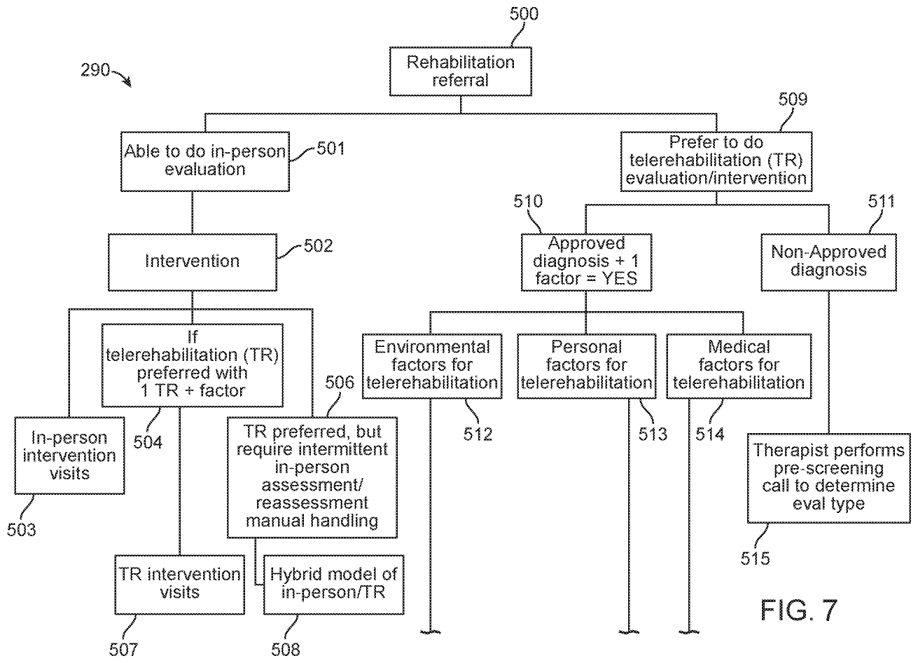

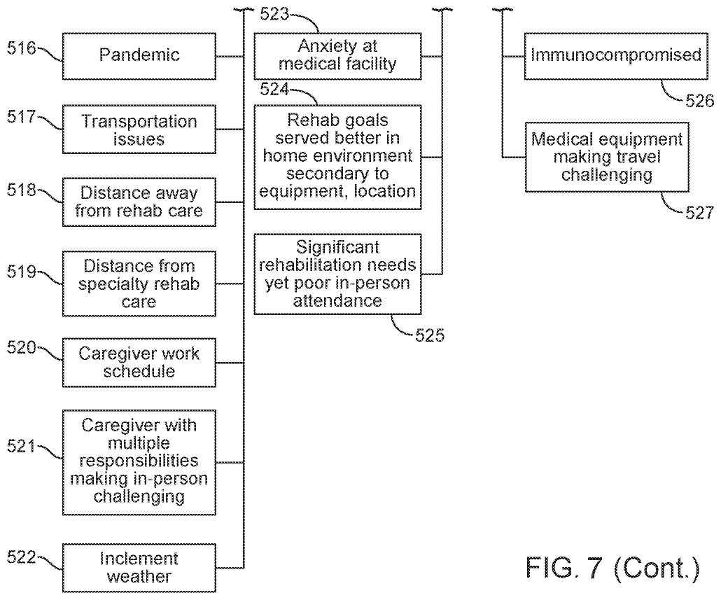

Turning now to FIG. 7, an example logic flow diagram is shown generally at 290 for determining how to handle telemedicine visits for patients requiring physical therapy (PT) and/or occupational therapy (OT) in the inpatient setting during times other than a pandemic or health crisis. The process begins at block 500 where there a rehabilitation referral occurs. At block 501 if an in-person evaluation is possible, an intervention is performed at block 502. Intervention includes in-person intervention visits at block 503, telerehabilitation preferred visits at block 504, and telerehabilitation preferred but intermittent in-person assessments and/or reassessments including manual handling at block 506. At block 507 if telerehabilitation was preferred, then a telerehabilitation intervention visit is provided. If telerehabilitation was preferred at block 506, then a hybrid model of in person and telerehabilitation is continued at block 508.

The process continuing from block 509 occurs if the patient prefers to do telerehabilitation evaluation and intervention. First at block 510, it is determined whether environmental factors for tele-rehabilitation exist at block 512; personal factors exist at block 513; and medical factors for tele-rehabilitation exist at block 514. In the event that the environmental factors exist for telerehabilitation, at block 512 then the following possible factors are reviewed: is there a pandemic (block 516); are there transportation issues (block 517); is the patient a distance away from the rehabilitation care center or office (block 518); is there a distance involved from a specialty rehabilitation care facility (block 519); are there issues with the family or other caregiver work schedule (block 520); does the family have multiple responsibilities making in-person visits challenging (block 521); and is inclement weather a factor (block 522).

If there are personal factors involved when determining if telerehabilitation is appropriate at block 513, then additional factors for review include: does the patient have anxiety at medical facilities (block 523); are rehab goals served better in a home environment secondary to equipment location (block 524); and are there significant rehabilitation needs yet poor in-person attendance (block 525).

If there are medical factors for rehabilitation at block 514, then additional factors may include: is the patient immunocompromised (block 526) and does medical equipment make travel challenging (block 527). Returning to block 509, in the event that a non-approved diagnosis occurs at block 511, then the process moves to block 515 where a therapist performs a prescreening call to determine the evaluation type.

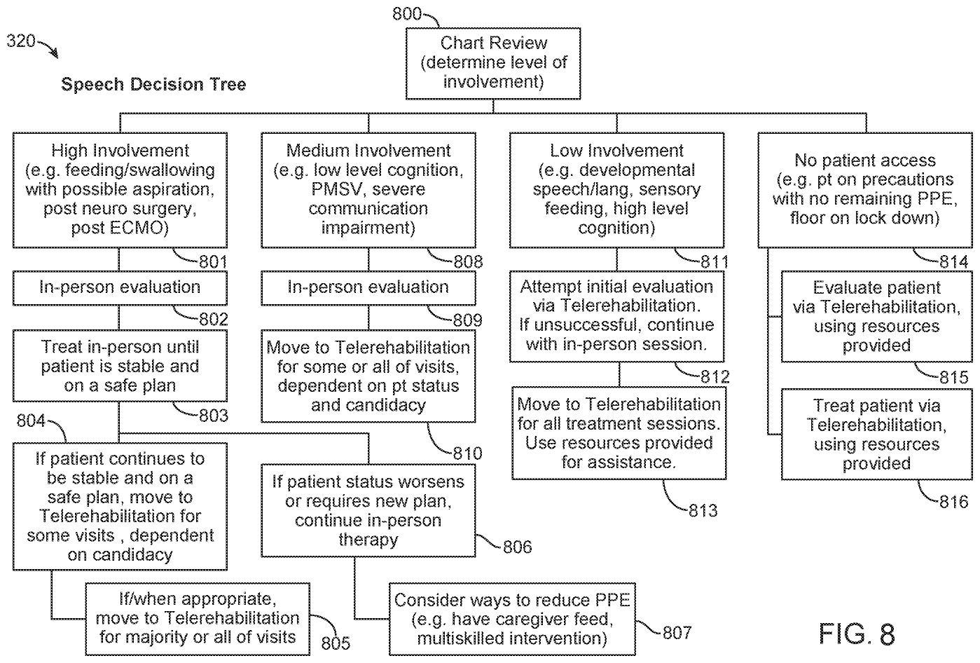

Turning next to FIG. 8, an example logical flow diagram is illustrated that provides a possible series of steps for determining how to handle telemedicine visits for patients requiring speech therapy. The process is shown generally at 320 and begins at block 800 where the chart of a patient is reviewed to determine the level of involvement. In the case of high involvement at block 801 (e.g., feeding/swallowing with possible aspiration, post-neurosurgery, post ECMO). An in-person evaluation is next performed a block 802 and the process continues to block 803 where the patient is treated in-person until the patient is stable and on a safe plan. Moving to block 804, if the patient continues to be stable and on a safe plan, telerehabilitation may be introduced for some visits depending on the candidacy. Finally, at block 805, if and when appropriate the patient may move to telerehabilitation for majority of all some or all visits. If from block 803 the patient status worsens or requires a new plan, then at block 806 in-person therapy is continued. The process continues to block 807 to consider ways to reduce PPE (e.g., have the caregiver feed, multiskilled intervention, etc.).

Still referring to FIG. 8, if a medium involvement is determined at block 808, such involvement may include low level cognition, PMSV, or severe communication impairment. In such instances an in-person evaluation is performed at block 809 and proceeds to block 810 where movement to telerehabilitation for some or all visits depending on the physical therapy status and candidacy are determined. If low speech or language, sensory feeding, or a high-level cognition is determined at block 811, the process proceeds to block 812 to attempt an initial evaluation via telerehabilitation. If the attempt is unsuccessful in-person sessions are continued. The process then moves to block 813 to move to tele-rehabilitation for all treatment session using resources provided for assistance.

If no patient access is available at block 814 (for example due to physical therapist precautions, no remaining PPE, floor or building is on lockdown, or other factors), then the process moves to block 815 to evaluate the patient via telerehabilitation using available resources. Finally, the process continues to block 816 to treat the patient via telerehabilitation using the resources provided.

Table 5 below provides a prophetic example of a low complexity situation in which speech therapy may be provided via telerehabilitation.

TABLE 5
Case example:
Baby seen on CV, demonstrating
developmental and speech/language delays.
Categorized as “low complexity”
Attempt initial evaluation via telerehabilitation,
gathering information formally
(e.g. REEL-3 parent questionnaire)
or informally (e.g. clinical observation)
If the telerehabilitation evaluation was
unsuccessful or incomplete, continue with
in-person evaluation session. This may
occur if caregiver has difficulty
understanding questions or is a poor reporter.
Plan to conduct treatment sessions via
Telerehabilitation, with a focus on parent/
caregiver coaching and education, along
with demonstration of intervention strategies

Table 6 below includes a checklist and issues that may be considered when providing outpatient telerehabilitation.

TABLE 6
Outpatient Telerehabilitation Checklist
Verbal consent for Telerehabilitation each session
Ask physical location and phone number of patient
in case of disconnection or emergency
Time started and time ended needs to be recorded
May want to log time outside of video system in
case of technology issues
1. First Session
Status check-in
Technology education and trouble-shooting
Feasibility of Telerehabilitation
during COVID-19
During current family life situation (e.g.,
work schedules, other family
members to care for)
Survey of supplies
Space
Quiet
Siblings
Lighting
Stating expectations for caregiver
Review caregiver's preferred learning style
Verbal
Printed documents
Video
Physical demonstration
Comfort with caregiver/child
therapeutic relationship
Handling
Coaching
Therapeutic activities
Plan for next session
Time
Equipment
Goals
2. Ongoing Sessions
Status check-in
Symptoms
Progress
Questions from last session
HEP update
Concerns for day's session
Plan for session
Activities
Equipment
Space
Camera position
Therapeutic activities
Demonstrate/explain first
especially if new activity
Physical demonstration
Doll
Video/image
Practice together
Observe
Reflect
How did that go?
Therapist gives feedback
Joint planning for next session
Activities, space, equipment/
materials, date/time

Table 7 below includes a checklist and issues that may be considered when providing outpatient telerehabilitation for various types of rehabilitation services.

TABLE 7

| | |
|---|---|
| General | Caregiver presence mandatory throughout the appointment |
| Recommendations | Ensure that patient and caregiver are in a quiet area with |
| | minimal background noise during testing |
| | If possible, send out speech/language questionnaire and informal evaluation |
| | handouts to family before the visit (via patient portal's, email, fax, or mail), |
| | requesting all items returned at least 24 hours before the appointment |
| | Instruct caregivers (and interpreters, if relevant) not to provide any |
| | answers/clues to the patient during standardized testing |
| | Document any testing modifications for standardized assessments |
| | Reach out to school team if patient has already been assessed by the district |
| Speech | Hold up paper stimulus book for Goldman Fristoe to camera, or access for |
| articulation & | free via QGlobal |
| Phonological | Ensure a clear view of the patient's face while they are producing sounds |
| Disorders | Consider use of headphones, request that family use an external microphone, |
| | if available |
| | If unable to complete standardized testing, use a speech sample and caregiver |
| | report |
| | Ask caregivers to report accuracy of speech sounds, using a rating scale |
| | Request that caregiver record and send a speech sample if patient is |
| | too shy in front of the camera |
| Receptive & | Consider use of parent questionnaires (e.g. REEL-3) for younger children |
| Expressive | Use informal measures (e.g. language sample, picture description, direction |
| Language | following in semi-structured play, MLU, etc.) |
| Disorders | Free resources available on QGlobal including stimulus books for CELF-5 |
| | and CELF-5 Spanish |
| Cognitive | Offer frequent breaks during testing, especially if patient is sensitive to |
| Communication | screens |
| Deficits | Suggest that patient/caregiver dim the computer screen and look away when |
| | possible |
| | Refer to Neuropsych or school testing, if available |
| Feeding & | Schedule appointment during a regular mealtime to observe a typical feeding |
| Swallowing | Request that family complete a food log or the “Food Checklist” prior to the |
| Disorders | visit |
| | Family may need to move camera to allow for different views throughout |
| | feeding, as well as a complete seating assessment |
| | If possible, provide caregiver some instruction on which foods to have |
| | prepared, before the appointment |
| Fluency | Request videos and examples of dysfluencies from everyday conversation |
| Disorders | Use informal assessment measures, including game play or a conversation |
| | sample with a familiar communication partner |
| | Instruct parents not to interrupt the patient's speech or complete their |
| | sentences during testing |
| Augmentative & | Use informal testing measures, including clinical observation with as many |
| Alternative | communication partners as possible, use of highly motivating toys/activities |
| Communication | Request videos from family, from a variety of different settings |
| | Fill out an ROI for SGD trial loaner devices; refer to company websites and |
| | reps for current options for loaning out devices |

Table 8 provides a summary of tips for particular situations that can arise when doing physical therapy telerehabilitation.

TABLE 8

| Evaluation | Caregiver presence mandatory unless adult age |
|---|---|
| Torticollis | Assessment:-Use screen shots to measure resting tilt, A/PROM or could use |
| | bony landmarks to estimate ROM |
| | Developmental assessment: AIMS |
| | Preparation:-Use handouts or demonstration with doll to demonstrate position |
| | to family |
| | Tech: Use Snip (IT can upload), or print screen function (different per |
| | computer) |
| Concussion | A: Cervical ROM-screen shot, Vestibular assessment based on reported |
| | symptoms, Modified BESS (in a corner or with supervision), body weight |
| | exercises/cardio in place w/ RPE |
| | P: RPE scale |
| | T: Screen share black screen |
| Ortho | A: Caregiver/patient palpate for pain, do MMT between grade 3 and 4 by |
| | caregiver, special tests that don't require manual (Ely test, impingement), |
| | functional strength tests |
| | P: Patient reported outcome related to joint, Functional disability inventory |
| | T: Screenshot ROM |
| Acute Pain | A: Functional disability inventory, functional strength tests, 4 S (school, sport, |
| (non ortho) | sleep, social life), referral to pain clinic |
| | P: Pain scale (mFACES, numeric) |
| | T: |
| Cancer | A: 30 sec sit to stand, SLS, calf raises, MMT (3 or 4), Observational Gait Scale |
| | (OGS), PedsQL, PROMIS scales, functional strength, dorsiflexion wall test |
| | (measure distance from wall) |
| | P: Send questionnaires if possible |
| | T: Consider hallway view for gait analysis (sagittal and frontal view) |
| IP to OP | A: ADL assessment screening tools, caregiver assistance important, |
| transfer | if safety issue consider home health or in-person |
| (trauma) | Assessment: Steps to home tool under G:\Rehab\Inpatient |
| | Documentation-Mpls\Steps to home |
| | P: Send questionnaires if possible by MyChart |
| | T: Need to position the phone prior to activity to allow safe caregiver assist |

As discussed above in connection with FIGS. 1-4, in connection with telerehabilitation, patient computing device 108 may be used by the patient; the patient may wear various sensors 120 during sessions; gamification and other age appropriate features may be included; and the system 100 and telemedicine server 102 may provide the functionality as described above.

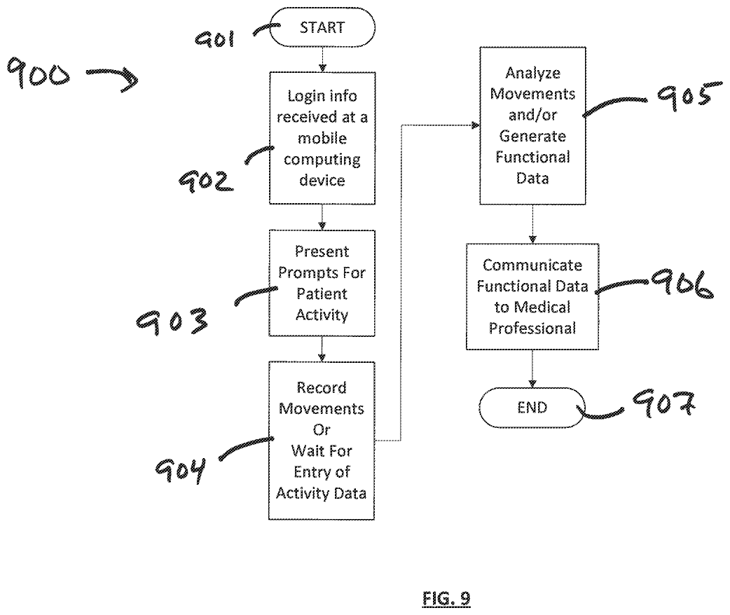

Next referring to FIG. 9, a logical flow diagram illustrating method steps which may be used in connection with the present invention is shown generally at 900. The method starts at 901 and continues to block 902 where log-in information is received at a computing device (for example patient computing device 108 in FIG. 1). The process moves to block 903 where prompts are presented on the computing device for the patient to begin activity. Next at block 904, movements may be recorded by video capture by the computing device and/or by sensors 120. Alternatively, the patient may manually enter data about the physical activity that was conducted. At block 905 the captured data and manually entered data is analyzed and functional data is generated. Moving to block 906, the data is communicated to the medical professional. In addition, at least a portion of the data may be communicated to a family or caregiver computing device 170 in the form of a status dashboard 180 or other graphical user interface. The process then ends at 907.

Although various embodiments are described herein, those of ordinary skill in the art will understand that many modifications may be made thereto within the scope of the present disclosure. Accordingly, it is not intended that the scope of the disclosure in any way be limited by the examples provided.

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components, unless context requires otherwise.

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

1. A system for providing functional testing and training of a patient, the system comprising:
at least one patient computing device comprising a processor and a memory comprising instructions that, when executed, operate a functional testing application including:
a) a functional testing service configured to manage a telerehabilitation functional testing session with the patient;
b) a goal setting service configured to create and monitor functional fitness goals for the patient;
c) a home exercise service configured to guide the patient through exercises selected for the patient;
d) a progress reporting service configured to track data collected by the functional testing service and the home exercise service to track rehabilitation progress of the patient; and
e) a provider communication service configured to facilitate communication between the patient and a healthcare provider.

2. The system of claim 1, further comprising one or more sensors configured to be worn by the patient, the one or more sensors operating to record movements of the patient and transmit data regarding the movements to the at least one patient computing device.

3. The system of claim 1, wherein the provider communication service is further configured to facilitate communication with a proxy of the patient.

4. A non-transitory computer-readable storage medium comprising computer-executable instructions which, when executed by a computing system, cause the computing system to perform a method of functional testing of a subject, the method comprising:
receiving login information from the subject at a mobile computing device;
presenting prompts on the mobile computing device to have the subject perform one or more actions;
recording subject movements with the mobile computing device;
analyzing movements to provide functional data regarding the subject's physical function capacity; and
communicating the functional data to a computing device operated by an observing professional.

5. The non-transitory computer-readable storage medium of claim 4, wherein the subject movements are recorded using one or more sensors.

6. The non-transitory computer-readable storage medium of claim 5, wherein the sensor is an accelerometer.

7. The non-transitory computer-readable storage medium of claim 4, wherein the movements are analyzed by comparing the subject movements to standard movements corresponding to the prompts.

8. The non-transitory computer-readable storage medium of claim 6, wherein the movements are analyzed in real time as the subject performs the movements.

9. The non-transitory computer-readable storage medium of claim 4, wherein the subject is a pediatric patient and the professional is a physical therapist.

10. A method of functional testing of a subject between in-person sessions, the method comprising:
receiving login information from the subject at a mobile computing device;
presenting prompts on the mobile computing device to have the subject perform one or more actions;
recording subject movements with the mobile computing device;
analyzing movements to provide functional data regarding the subject's physical function capacity; and
communicating the functional data to a computing device operated by an observing professional.

11. The method of claim 10, wherein the subject movements are recorded using one or more sensors.

12. The method of claim 11, wherein the sensor is an accelerometer.

13. The method of claim 10, further comprising comparing the subject movements to standard movements corresponding to the prompts.

14. The method of claim 13, further comprising analyzing in real time as the subject performs the movements.

15. The method of claim 10, wherein the subject is a pediatric patient and the professional is a physical therapist.

16. The method of claim 15, wherein at least a portion of the functional data is provided in a dashboard to a caregiver computer device.

17. A telerehabilitation system comprising:
at least one patient computing device comprising a video conferencing application configured to receive telerehabilitation services;
at least one telemedicine provider computing device configured to operate a video conferencing application to communicate with the at least one patient computing device to provide telerehabilitation services;
a telemedicine server comprising:
a graphical user interface operable to present a visual display on the at least one patient computing device and at least one telemedicine provider computing device for facilitating telerehabilitation sessions;
a communication module operable to manage communication between the at least one patient computing device, the at least one telemedicine provider computing device, and the telemedicine server;
a telerehabilitation services manager operable to coordinate telerehabilitation services; and
a data store comprising telerehabilitation resources accessible by the at least one patient computing device and the at least one telemedicine provider computing device.

18. The telerehabilitation system of claim 17, wherein the telerehabilitation sessions are conducted between in-person sessions.

19. The telerehabilitation system of claim 17, wherein the telemedicine provider computing device is arranged and configured to provide telerehabilitation sessions with a plurality of patient computing devices.

20. A system for providing remote rehabilitation therapy, comprising:
one or more sensors configured to be worn by a patient, the sensors comprising:
movement sensors configured to record motion data from inertial and/or mechanomyographic movements of a patient during an activity; and
a wireless communication device;
a computing device comprising:
a processing device; and
a memory device comprising instructions which, when executed by a computing device, cause the computing system to:
receive, via wireless communication, the motion data recorded by the sensors;
communicate the motion data to a computing device operated by a medical professional.
